(19) United States
(12) Patent Application Publication
(10) Pub. No.: US 2015/0147317 A1
(43) Pub. Date: May 28, 2015

(54) METHODS RELATED TO BEVACIZUMAB

(71) Applicant: MOMENTA PHARMACEUTICALS, INC., Cambridge, MA (US)

(72) Inventors: John Robblee, Newton, MA (US); Brian Edward Collins, Arlington, MA (US); Ganesh Kaundinya, Bedford, MA (US); Carlos J. Bosques, Arlington, MA (US)

(21) Appl. No.: 14/403,835

(22) PCT Filed: May 31, 2013
(86) PCT No.: PCT/US13/43696

§ 371 Date: Nov. 25, 2014

Related U.S. Application Data
(60) Provisional application No. 61/654,523, filed on Jun. 1, 2012.
(60) Provisional application No. 61/783,187, filed on Mar. 14, 2013.

(51) Int. Cl. G01N 33/68; C07K 16/22

(52) U.S. Cl. CPC G01N 33/6854; C07K 16/22; C07K 2317/24; G01N 2440/38; G01N 2560/00
USPC 424/133.1; 530/387.3; 435/69.6

(57) ABSTRACT

The present invention relates to the characterization and production of bevacizumab.

This application claims the benefit of U.S. Provisional Application No. 61/654,523, filed Jun. 1, 2012; and U.S. Provisional Application 61/783,187, filed Mar. 14, 2013.

This disclosure provides compositions and methods related to bevacizumab.

### BACKGROUND OF THE INVENTION

Bevacizumab (Avastin®) is a recombinant humanized monoclonal IgG1 antibody that binds to and inhibits the biologic activity of human vascular endothelial growth factor (VEGF) in in vitro and in vivo assay systems. Bevacizumab contains human framework regions and the complementarity-determining regions of a murine antibody that binds to VEGF. Bevacizumab has an approximate molecular weight of 149 kD.

Bevacizumab is presently indicated for the treatment of (i) metastatic colorectal cancer, with intravenous 5-fluorouracil-based chemotherapy for first- or second-line treatment; (ii) non-squamous non-small cell lung cancer, with carboplatin and paclitaxel for first line treatment of unresectable, locally advanced, recurrent or metastatic disease; (iii) metastatic breast cancer, with paclitaxel for treatment of patients who have not received chemotherapy for metastatic HER2-negative breast cancer; (iv) glioblastoma, as a single agent for adult patients with progressive disease following prior therapy; (v) metastatic renal cell carcinoma with interferon-alpha (from Avastin® Prescribing Information dated Dec. 21, 2011, Genentech, Inc.).

### SUMMARY OF THE INVENTION

The present disclosure provides, in part, methods for evaluating, identifying, and/or producing (e.g., manufacturing) bevacizumab. In some instances, methods herein allow highly resolved evaluation of bevacizumab useful for, inter alia, manufacturing bevacizumab, characterizing bevacizumab, identifying and/or confirming bevacizumab, monitoring the structure of bevacizumab, comparing bevacizumab preparations made over time or made under different conditions, and/or controlling the structure of bevacizumab.

In certain aspects, the disclosure provides methods of evaluating a glycoprotein preparation (e.g., such as a glycoprotein drug substance or drug product preparation). Such methods can include evaluating the glycoprotein preparation for the presence, absence, level and/or ratio of one or more (e.g., two or more when working with ratios) bevacizumab-specific parameters (i.e., acquiring information (e.g., value(s)) pertaining to the bevacizumab-specific parameters). Such methods can also optionally include providing, e.g., acquiring, a determination of whether the presence, absence, level and/or ratio of one or more bevacizumab-specific parameters evaluated meets a reference criteria for the one or more bevacizumab-specific parameters, which determination includes, for example, comparing the presence, absence, level and/or ratio of one or more bevacizumab-specific parameters evaluated with the reference criteria and/or confirming that the presence, absence, level or ratio of one or more bevacizumab-specific parameters evaluated has a defined (e.g., predefined) relationship with the reference criteria. In some instances, the one or more (e.g., two or more when working with ratios) bevacizumab-specific parameters evaluated include one or more (e.g., 1, 2, 3, 4, 5, 6, 7, 8, 9, 10, 11, 12, 13, 14, 15) parameters disclosed in Table 1.

In certain other aspects, the disclosure provides methods of manufacturing bevacizumab drug product, such methods include a first step of providing (e.g., producing or expressing (e.g., in small scale or large scale cell culture) or manufacturing) or obtaining (e.g., receiving and/or purchasing from a third party (including a contractually related third party or a non-contractually-related (e.g., an independent) third party) a test glycoprotein preparation (e.g., a sample of a test glycoprotein preparation), a second step of acquiring (e.g., detecting, measuring, receiving, or obtaining, as discussed subsequently herein) at least one value (e.g., 1, 2, 3, 4, 5, 6, 7, 8, 9, 10, 11, 12, 13, 14, 15, 16, 17, 18, 19, 11, 12, 13, 14, or 15) for a bevacizumab parameter listed in Table 1 for the test glycoprotein preparation, and a third step of processing at least a portion of the test glycoprotein preparation (e.g., processing a portion of a manufacturing lot, batch, or run, an entire manufacturing lot, batch, or run, or multiple manufacturing lots, batches, or runs) as bevacizumab drug product (e.g., in a form or packaging intended for marketing or administration as described subsequently herein) if the at least one value for the test glycoprotein preparation meets a reference criterion shown in Table 1 for the parameter, thereby manufacturing bevacizumab drug product. In some instances, the second step of such methods includes acquiring values for any combination of two or more bevacizumab parameters listed in Table 1, and the third step of such methods includes processing at least a portion of the test glycoprotein preparation as bevacizumab drug product if the values for the any combination of two or more bevacizumab parameters for the test glycoprotein preparation meet the corresponding reference criterion shown in Table 1 for the parameters. In some instances, the any combination of two or more bevacizumab parameters can include 2, 3, 4, 5, 6, 7, 8, 9, 10, 11, 12, 13, 14, or 15 of the bevacizumab parameters listed in Table 1 and/or any two or more of parameter numbers 1, 2, 3, 4, 5, 6, 7, 8, 9, 10, 11, 12, 13, 14, 15, 16, 17, 18, 19, 11, 12, 13, 14, and/or 15 shown in Table 1. In some instances, the second step of such methods includes acquiring a value for a plurality of bevacizumab parameters listed in Table 1, and the third step of such methods includes processing at least a portion of the test glycoprotein preparation as bevacizumab drug product if the value for the plurality for the test glycoprotein preparation meets the corresponding reference criterion shown in Table 1 for the parameters. In some instances, the plurality includes 2, 3, 4, 5, 6, 7, 8, 9, 10, 11, 12, 13, 14, or 15 of the bevacizumab parameters listed in Table 1 and/or parameter numbers 1, 2, 3, 4, 5, 6, 7, 8, 9, 10, 11, 12, 13, 14, and/or 15 shown in Table 1. In some instances, the second step of such methods includes acquiring a value for at least one value of bevacizumab parameters listed in Table 1, and the third step of such methods includes processing at least a portion of the test glycoprotein preparation as bevacizumab drug product if at least one of the at least one value for the plurality for the test glycoprotein preparation meets the corresponding reference criterion shown in Table 1 for the parameter.

In some instances, the test glycoprotein preparation obtained or produced in the first step of such methods includes a recombinant antibody composition having a first amino acid sequence with at least 85% identity to SEQ ID NO:1 (e.g., 90, 95, 98, or 100% identity to SEQ ID NO:1) and a second amino acid sequence with at least 85% identity to SEQ ID NO:2 (e.g., 90, 95, 98, or 100% identity to SEQ ID NO:2). In some instances, the recombinant antibody composition includes a first amino acid sequence with 100% identity to SEQ ID NO:1 and a second amino acid sequence with 100% identity to SEQ ID NO:2. In either instance, the first and second amino acid sequence combine when expressed to form the recombinant antibody in which the first sequence is the antibody heavy chain and the second sequence is the antibody light chain.

In some instances, evaluation methods include, for a glycoprotein preparation, evaluating information (e.g., value(s)) pertaining to one or more bevacizumab-specific parameters and, optionally, providing, e.g., acquiring, a determination of whether the information meets a bevacizumab signature, e.g., by comparing the information with the bevacizumab signature and/or confirming that the information has a defined (e.g., predefined) relationship with the bevacizumab signature.

In some instances, evaluation methods include, for a glycoprotein preparation, evaluating information (e.g., value(s)) pertaining to one or more of the bevacizumab parameters disclosed in Table 1, and, optionally, providing, e.g., acquiring, a determination of whether the information meets a bevacizumab signature, e.g., by comparing the information with the bevacizumab signature and/or confirming that the information has a defined (e.g., predefined) relationship with the bevacizumab signature. For example, for a given glycoprotein preparation, methods can include: evaluating HM5 and obtaining a value therefor, and, optionally, determining whether the value conforms to the reference criterion for HM5 provided in Table 1, wherein, in this example, the reference criterion for HM5 is a bevacizumab signature. In this instance, the value for HM5 would conform to the bevacizumab signature if it is less than 0.09.

In another aspect, the disclosure provides methods of identifying a test glycoprotein preparation (e.g., such as a glycoprotein drug substance or drug product preparation) as bevacizumab. In some instances, identification methods include, for a glycoprotein preparation, evaluating information (e.g., value(s)) pertaining to one or more bevacizumab-specific parameters, providing, e.g., acquiring, a determination of whether the information meets a bevacizumab signature, e.g., by comparing the information with the bevacizumab signature and/or confirming that the information has a defined (e.g., predefined) relationship with the bevacizumab signature, and identifying the glycoprotein preparation as bevacizumab if the information meets the bevacizumab signature.

In some instances, identification methods include, for a glycoprotein preparation, evaluating information (e.g., value(s)) pertaining to one or more of the ‘bevacizumab parameters’ disclosed in Table 1, providing, e.g., acquiring, a determination of whether the information meets a bevacizumab signature, e.g., by comparing the information with the bevacizumab signature and/or confirming that the information has a defined (e.g., predefined) relationship with the bevacizumab signature, and identifying the glycoprotein preparation as bevacizumab if the acquired information meets the bevacizumab signature. For example, for a given glycoprotein preparation, methods can include: evaluating HM5 and obtaining a value therefor, determining whether the value conforms to the reference criterion for HM5 provided in Table 1, and identifying the glycoprotein preparation as bevacizumab if the information conforms, wherein, in this example, the reference criterion for HM5 is a bevacizumab signature. In this instance, the value for HM5 would conform to the bevacizumab signature if it is less than 0.09.

In a further aspect, the disclosure provides methods of producing (e.g., manufacturing) bevacizumab (e.g., bevacizumab drug product). In some instances, production methods include, for a glycoprotein preparation, evaluating information (e.g., value(s)) pertaining to one or more bevacizumab-specific parameters, providing, e.g., acquiring, a determination of whether the information meets a bevacizumab signature, e.g., by comparing the information with the bevacizumab signature and/or confirming that the information has a defined (e.g., predefined) relationship with the bevacizumab signature, and processing the glycoprotein preparation (e.g., as bevacizumab drug product) if the information meets the bevacizumab signature, thereby producing bevacizumab (e.g., bevacizumab drug product). In some instances, production methods include, for a glycoprotein preparation, evaluating information (e.g., value(s)) pertaining to one or more bevacizumab parameters disclosed in Table 1, providing, e.g., acquiring, a determination of whether the information meets a bevacizumab signature, e., by comparing the information with the bevacizumab signature and/or confirming that the information has a defined (e.g., predefined) relationship with the bevacizumab signature, and processing the glycoprotein preparation (e.g., as bevacizumab drug product) if the information meets the bevacizumab signature, thereby producing bevacizumab (e.g., bevacizumab drug product). For example, for a given glycoprotein preparation, production methods can include: evaluating a value for HM5 for the glycoprotein preparation, comparing the value with the reference criterion for HM5 provided in Table 1, determining whether the value obtained meets with the reference value for HM5, and processing the glycoprotein preparation as bevacizumab drug product if the value obtained meets the reference criterion for HM5, wherein, in this example, the reference criterion for HM5 is a bevacizumab signature. In this instance, the value for HM5 would conform to the reference criterion for HM5 if it is less than 0.09. In some instances, these methods can further include packaging, labeling, and/or shipping the bevacizumab drug product, e.g., as discussed in further detail herein.

As used herein, a bevacizumab signature comprises a plurality of reference criteria or rules for a plurality of parameters that define bevacizumab. In some instances, a bevacizumab signature can be a pharmaceutical specification, a commercial product release specification, a product acceptance criterion, a pharmacopeial standard, or a product labeling description. In some instances, the bevacizumab signature comprises a plurality of reference criteria or rules for a plurality of parameters shown in Table 1:

TABLE 1

| | | Parameter | |
|---|---|---|---|
| Parameter # | Parameter Category | 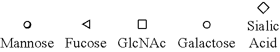 | Reference Criterion (rule) |
| 1 | HM5 | 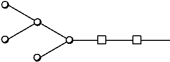 | <0.90%* |
| 2 | HM6 | 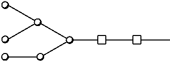 | <0.05%* |
| 3 | HM7 | 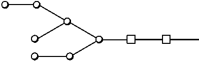 | <0.05%* |
| 4 | G0F | 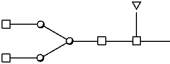 | >60.00%* |
| 5 | G1F | 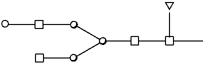 | <20.00%* |
| 6 | G1F | 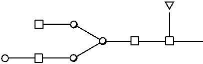 | <6.00%* |
| 7 | G2F | 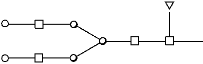 | <2.60%* |
| 8 | C-Terminal- | Amount of lysine present at the C-terminus of the | <5.00%^{$} |
| | lysine | heavy chain | |
| 9 | HC-Pyroglu | Pyroglutamate (pyroglu) at the N-terminus of the | <10.00%^{#} |
| | | heavy chain | |
| 10 | LC-Pyroglu | Pyroglutamate at the N-terminus of the light | <3.00%^{#} |
| | | chain | |
| 11 | HC-M256- | Post-translational modification of the M256 | >4.00%^{#} |
| | Sulfo | residue (Kabat et al. numbering) of the heavy | |
| | | chain-residue is oxidized to form methionine | |
| | | sulfoxide | |
| 12 | LC-K149-Glyc | Post-translational glycation at lysine 149 of the | >0.50%^{#} |
| | | light chain | |
| 13 | LC-135 | Amount of free cysteine (e.g. not paired in | <1.20%\hat{ } |
| | | disulfides) at cysteine 135 in the light chain | |
| 14 | HC148 | Amount of free cysteine (e.g. not paired in | <10.00%\hat{ } |
| | | disulfides) at cysteine 148 in the heavy chain | |
| 15 | HC204 | Amount of free cysteine (e.g. not paired in | <5.00%\hat{ } |
| | | disulfides) at cysteine 204 in the heavy chain | |

*For any given parameter, percent refers to the number of moles of PNGase F-released glycan X relative to total moles of PNGase F-released glycan detected as disclosed in Table 2, wherein X represents the parameter of interest (e.g., parameter(s) 1-7).
^{#}For any given parameter, percent refers to the level of modified peptide Y relative to the sum of the levels of modified peptide Y and unmodified peptide Y, detected as disclosed in Table 2, wherein Y represents the parameter of interest (e.g., parameter(s) 9-12).
^{$}For C-terminal-lysine, percent refers to the level of C-terminal-lysine-containing peptide relative to the sum of the levels of C-terminal-lysine-containing and C-terminal-lysine-free peptides detected as disclosed in Table 2.
\hat{ }For free cysteine, percent refers to the level of non-disulfide-linked peptide relative to the sum of the levels of non-disulfide-linked and disulfide-linked peptides, detected as disclosed in Table 2.

While the present disclosure provides exemplary units and methods for the evaluation, identification, and production methods disclosed herein (see, e.g., Tables 1 and 2), a person of ordinary skill in the art will appreciate that performance of the evaluation, identification, and production methods herein is not limited to use of those units and/or methods. For example, bevacizumab signatures described herein are generally described, for each parameter, as a value for a glycan or structure relative to total glycan on a mol/mol basis (see, e.g., Table 1). A person of skill in the art understands that although the use of other metrics or units (e.g., mass/mass, mole percent vs. weight percent) to measure a described parameter might give rise to different absolute values than those described herein, e.g., in Table 1, a test glycoprotein preparation meets a disclosed bevacizumab reference criterion or signature even if other units or metrics are used, as long as the test glycoprotein preparation meets the herein disclosed reference criterion or signature when the herein disclosed units and metrics are used, e.g., allowing for the sensitivity (e.g., analytical variability) of the method being used to measure the value.

Bevacizumab parameters shown in Table 1 are parameters that, alone, in any combination, or together, distinguish bevacizumab from non-bevacizumab glycoprotein (see below). In some instances, a bevacizumab parameter is part of the glycoprotein, e.g., connected with the rest of the glycoprotein by a covalent bond, i.e., an intrinsic parameter. Intrinsic parameters include the presence, absence, level, ratio (with another entity), or distribution of a physical moiety, e.g., a moiety arising from or associated with a post-translational event. Exemplary parameters include the presence (or absence), abundance, absolute or relative amount, ratio (with another entity), or distribution of a glycan, a linkage, a glycoform, or post-translationally added components of the preparation. In some instances, a parameter is not part of the glycoprotein but is present in the preparation with the glycoprotein (i.e., in a glycoprotein preparation), i.e., an extrinsic, parameter. Exemplary parameters of this type include the presence (or absence), abundance, ratio (with another entity), or distribution of, e.g., impurities, e.g., host cell proteins, residue from purification processes, viral impurities, and enclosure components.

In some instances, a bevacizumab signature comprises reference criteria or rules for 2, 3, 4, 5, 6, 7, 8, 9, 10, 11, 12, 13, 14, 15, or substantially all, parameters shown in Table 1. In some instances, a bevacizumab signature comprises reference criteria or rules for two or more (e.g., 2, 3, 4, 5, 6, 7, 8, 9, 10, 11, 12, 13, 14, or 15) of bevacizumab parameter(s) 1, 2, 3, 4, 5, 6, 7, 8, 9, 10, 11, 12, 13, 14, and/or 15. In some instances, a bevacizumab signature comprises predetermined reference criteria or rules for 2, 3, 4, 5, 6, 7, 8, 9, 10, 11, 12, 13, 14, or 15 parameters shown in Table 1. In some instances, predetermined reference criteria can include reference criterion or criteria for: parameter number(s) 1, 2, and/or 3 shown in Table 1; parameter number(s) 1 and/or 2 from Table 1; parameter number(s) 1, 2, 3, and/or 4 from Table 1; parameter number (s) 1, 2, 3, 4, 6, 7, 13, or 14 from Table 1; parameter number (s) 1, 2, 3, 4, 5, 6, 7, 8, 13, 14, and/or 15 from Table 1; a combination of one or more (e.g., two or three) of parameter number(s) 1, 2, and 3 with one or more (e.g., two, three, four, five, six, seven, eight, nine, ten, eleven, or twelve or more) of parameter number(s) 4, 5, 6, 7, 8, 9, 10, 11, 12, 13, 14, and 15; parameter number(s) 1 and 2 from Table 1 further combined with one or more (e.g., two, three, four, five, six, seven, eight, nine, ten, eleven, twelve, or thirteen) of the other parameter number(s) from Table 1; one or more (e.g., two) of parameter number(s) 1 and 2 from Table 1 combined with one or more (e.g., two) of parameter number(s) 3 and 4; two or more (e.g., three or four) of parameter number(s) 1, 2, 3 and 4 from Table 1, optionally further combined with one or more (e.g., two, three, four, five, six, seven, eight, nine, ten, or eleven) of the other parameter number(s) from Table 1; one or more of parameter number(s) 1, 2, 3, and 4 from Table 1 with one or more (e.g., two, three, four, five, six, seven, eight, nine, ten, or eleven) of the other parameter number(s) in Table 1; one or more (e.g., two, three or four) of parameter number(s) 1, 2, 3 and 4 from Table 1 combined with one or more (e.g., two, three or four parameter number(s) selected from the group consisting of parameter number(s) 6, 7, 13, and 14; a combination of two or more (e.g., three, four, five, six, seven, or eight) of parameter number(s) 1, 2, 3, 4, 6, 7, 13, and 14, optionally further combined with one or more (e.g., two, three, four, five, six, or seven) of the other parameter number(s) in Table 1; and/or one or more (e.g., three, four, five, six, seven, or eight) of parameter number(s) 1, 2, 3, 4, 6, 7, 13, and 14 combined with one or more (e.g., two or three) of parameter number(s) 5, 8, and 15.

In some instances, methods (i.e., evaluation, identification, and production methods) can further include, e.g., one or more of: providing or obtaining a glycoprotein preparation (e.g., such as a glycoprotein drug substance or a precursor thereof); memorializing confirmation or identification of the glycoprotein preparation as bevacizumab using a recordable medium (e.g., on paper or in a computer readable medium, e.g., in a Certificate of Testing, Certificate of Analysis, Material Safety Data Sheet (MSDS), batch record, or Certificate of Analysis (CofA)); informing a party or entity (e.g., a contractual or manufacturing partner, a care giver or other end-user, a regulatory entity, e.g., the FDA or other U.S., European, Japanese, Chinese or other governmental agency, or another entity, e.g., a compendial entity (e.g., U.S. Pharmacopoeia (USP)) or insurance company) that a glycoprotein preparation is bevacizumab; selecting the glycoprotein preparation for further processing (e.g., processing (e.g., formulating) the glycoprotein preparation as a drug product (e.g., a pharmaceutical product) if the glycoprotein preparation is identified as bevacizumab; reprocessing or disposing of the glycoprotein preparation if the glycoprotein preparation is not identified as bevacizumab.

In some instances, methods (i.e., evaluation, identification, and production methods) include taking action (e.g., physical action) in response to the methods disclosed herein. For example, the glycoprotein preparation is classified, selected, accepted or discarded, released or withheld, processed into a drug product, shipped, moved to a different location, formulated, labeled, packaged, released into commerce, or sold or offered for sale, depending on whether the preselected relationship is met.

In some instances, processing may include formulating, packaging (e.g., in a syringe or vial), labeling, or shipping at least a portion of the glycoprotein preparation. In some instances, processing includes formulating, packaging (e.g., in a syringe or vial), and labeling at least a portion of the glycoprotein as bevacizumab drug product. Processing can include directing and/or contracting another party to process as described herein.

### DEFINITIONS

As used herein, a glycoprotein refers to amino acid sequences that include one or more oligosaccharide chains (e.g., glycans) covalently attached thereto. Exemplary amino acid sequences include peptides, polypeptides and proteins. Exemplary glycoproteins include glycosylated antibodies and antibody-like molecules (e.g., Fc fusion proteins). Exemplary antibodies include monoclonal antibodies and/or fragments thereof, polyclonal antibodies and/or fragments thereof, and Fc domain containing fusion proteins (e.g., fusion proteins containing the Fc region of IgG1, or a glycosylated portion thereof). A glycoprotein preparation is a composition or mixture that includes at least one glycoprotein.

A glycoprotein preparation (e.g., such as a glycoprotein drug substance or a precursor thereof) included herein is or includes a glycoprotein (e.g., an antibody) that has a first amino acid sequence with at least 85% identity to SEQ ID NO:1 and a second amino acid sequence with at least 85% identity to SEQ ID NO:2. In some instances, the first and/or second amino acid sequence(s) have at least 90%, 91%, 92%, 93%, 94%, 95%, 96%, 97%, 98%, 99%, or 100% identity to SEQ ID NO:1 and/or at least 90%, 91%, 92%, 93%, 94%, 95%, 96%, 97%, 98%, 99%, or 100% identity to SEQ ID NO:2.

In some instances, a glycoprotein preparation (e.g., such as a glycoprotein drug substance or a precursor thereof) can be a sample from a proposed or test batch of bevacizumab drug substance or drug product. As used herein, a batch of a glycoprotein preparation refers to a single production run of the glycoprotein. Evaluation of different batches thus means evaluation of different production runs or batches. As used herein sample(s) refer to separately procured samples. For example, evaluation of separate samples could mean evaluation of different commercially available containers or vials of the same batch or from different batches.

As used herein, bevacizumab is the generic, compendial, nonproprietary, or official FDA name for the product marketed as Avastin® by Genentech/Roche Group and a product that is interchangeable with or equivalent to the product marketed as Avastin®.

As used herein, evaluating, e.g., in the evaluation/evaluating, identifying, and/or producing aspects disclosed herein means reviewing, considering, determining, assessing, analyzing, measuring, and/or detecting the presence, absence, level, and/or ratio of one or more bevacizumab-specific parameters in a glycoprotein preparation to provide information pertaining to the one or more bevacizumab-specific parameters. In some instances, evaluating can include performing a process that involves a physical change in a sample or another substance, e.g., a starting material. Exemplary changes include making a physical entity from two or more starting materials, shearing or fragmenting a substance, separating or purifying a substance, combining two or more separate entities into a mixture, performing a chemical reaction that includes breaking or forming a covalent or non-covalent bond. Evaluating can include performing an analytical process which includes a physical change in a substance, e.g., a sample, analyte, or reagent (sometimes referred to herein as “physical analysis”), performing an analytical method, e.g., a method which includes one or more of the following: separating or purifying a substance, e.g., an analyte, or a fragment or other derivative thereof, from another substance; combining an analyte, or fragment or other derivative thereof, with another substance, e.g., a buffer, solvent, or reactant; or changing the structure of an analyte, or a fragment or other derivative thereof, e.g., by breaking or forming a covalent or non-covalent bond, between a first and a second atom of the analyte; or by changing the structure of a reagent, or a fragment or other derivative thereof, e.g., by breaking or forming a covalent or non-covalent bond, between a first and a second atom of the reagent. In some instances, evaluating a glycoprotein preparation includes detecting the presence, absence, level or ratio of one or more (e.g., two or more when working with ratios) disclosed in Table 1 using methods disclosed in Table 2.

Information (e.g., value(s)) pertaining to a bevacizumab-specific parameter or a bevacizumab parameter means information, regardless of form, that describes the presence, absence, abundance, absolute or relative amount, ratio (with another entity), or distribution of a moiety associated with the glycoprotein preparation and/or bevacizumab. Information is evaluated in a glycoprotein preparation as disclosed herein. Information is also conveyed in a bevacizumab signature. Information can be qualitative, e.g., present, absent, intermediate, or quantitative, e.g., a numerical value such as a single number, or a range, for a parameter. In some instances, information is from a single sample or batch or a plurality of samples or batches. In some instances, information can be a range or average (or other measure of central tendency), e.g., based on the values from any X samples or batches, e.g., wherein at least of the samples or batches is being evaluated for commercial release, wherein X is equal to, at least, or no more than, 2, 3, 4, 5, 6, 7, 8, 9, 10, 11, 12, 13, 14 or 15. In some instances, information can be, for example: a statistical function, e.g., an average, of a number of values; a function of another value, e.g., of the presence, distribution or amount of a second entity present in the sample, e.g., an internal standard; a statistical function, e.g., an average, of a number of values; a function of another value, e.g., of the presence, distribution or amount of a second entity present in the sample, e.g., an internal standard; a value, e.g., a qualitative value, e.g., present, absent, “below limit of detection”, “within normal limits” or intermediate. In some instances, information can be a quantitative value, e.g., a numerical value such as a single number, a range of values, a “no less than x amount” value, a “no more than x amount” value. In some instances, information can be abundance. Abundance can be expressed in relative terms, e.g., abundance can be expressed in terms of the abundance of a structure in relation to another component in the preparation. E.g., abundance can be expressed as: the abundance of a structure (or a first group of structures) in Table 1 relative to the amount of protein; the abundance of a structure (or a first group of structures) in Table 1 relative to the abundance of a second structure (or second group of structures) in Table 1. Abundance, e.g., abundance of a first structure relative to another structure, can be with regard to the preparation as a whole, a single molecule, or a selected site on the protein backbone. E.g., the parameter can be the relative proportion of a first structure from Table 1 and a second structure from Table 1 at a selected site and the value can be expressed as, e.g., a proportion, ratio or percentage. Information can be expressed in any useful term or unit, e.g., in terms of weight/weight, number/number, number/weight, and weight/number. In many cases, the reference criterion is defined by a range of values.

As used herein, acquire or acquiring (e.g., acquiring information) means obtaining possession of a physical entity, or a value, e.g., a numerical value, by “directly acquiring” or “indirectly acquiring” the physical entity or value. Directly acquiring means performing a process (e.g., performing an assay or test on a sample or “analyzing a sample” as that term is defined herein) to obtain the physical entity or value. Indirectly acquiring refers to receiving the physical entity or value from another party or source (e.g., a third party laboratory that directly acquired the physical entity or value). Directly acquiring a physical entity includes performing a process, e.g., analyzing a sample, that includes a physical change in a physical substance, e.g., a starting material. Exemplary changes include making a physical entity from two or more starting materials, shearing or fragmenting a substance, separating or purifying a substance, combining two or more separate entities into a mixture, performing a chemical reaction that includes breaking or forming a covalent or non-covalent bond. Directly acquiring a value includes performing a process that includes a physical change in a sample or another substance, e.g., performing an analytical process which includes a physical change in a substance, e.g., a sample, analyte, or reagent (sometimes referred to herein as “physical analysis”), performing an analytical method, e.g., a method which includes one or more of the following: separating or purifying a substance, e.g., an analyte, or a fragment or other derivative thereof, from another substance; combining an analyte, or fragment or other derivative thereof, with another substance, e.g., a buffer, solvent, or reactant; or changing the structure of an analyte, or a fragment or other derivative thereof, e.g., by breaking or forming a covalent or non-covalent bond, between a first and a second atom of the analyte; or by changing the structure of a reagent, or a fragment or other derivative thereof, e.g., by breaking or forming a covalent or non-covalent bond, between a first and a second atom of the reagent. Exemplary analytical methods are shown in Table 2.

All literature and similar material cited in this application, including, but not limited to, patents, patent applications, articles, books, treatises, and web pages, regardless of the format of such literature and similar materials, are expressly incorporated by reference in their entirety. In the event that one or more of the incorporated literature and similar materials differs from or contradicts this application, including but not limited to defined terms, term usage, described techniques, or the like, this application controls. The section headings used herein are for organizational purposes only and are not to be construed as limiting the subject matter described in any way.

These, and other aspects of the invention, are described in more detail below and in the claims.

### DESCRIPTION OF THE DRAWINGS

FIG. 1|Amino acid sequence of heavy chain of bevacizumab (SEQ ID NO: 1).

FIG. 2|Amino acid sequence of light chain of bevacizumab (SEQ ID NO:2).

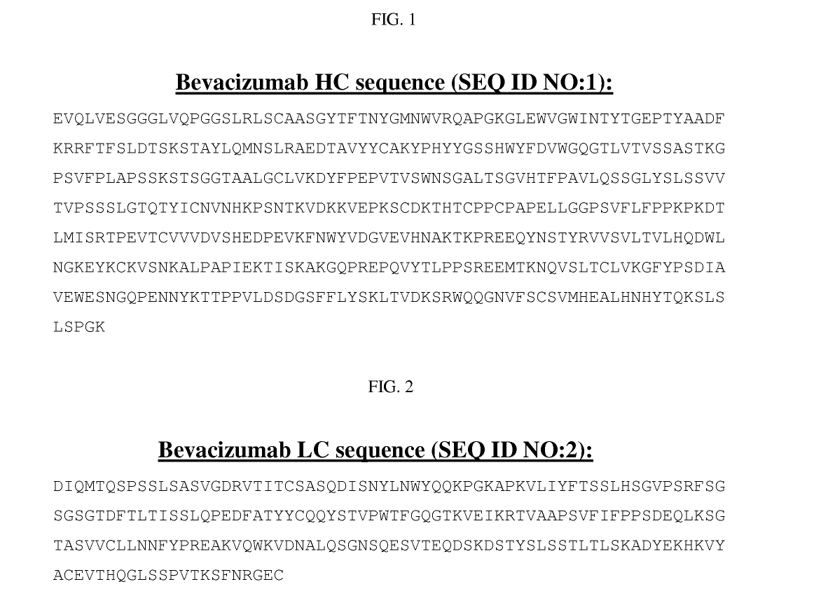

### DETAILED DESCRIPTION

Detailed, high resolution, structural information about Avastin® (e.g., related to the presence of signature glycan species or quantitative analyses ascribing site-specificity for backbone modifications) is useful to be able to make and test products that qualify as bevacizumab, e.g., that are interchangeable versions of Avastin®. Such information is also useful in monitoring product changes and controlling structural drift that may occur as a result of manufacturing changes. The art supports, however, that information necessary to be able to make and test products that qualify as bevacizumab, e.g., that are interchangeable versions of Avastin®, or any other branded biologic, is unavailable (see, e.g., Nowicki, “Basic Facts about Biosimilars,” Kidney Blood Press. Res., 30:267-272 (2007); Hincal “An Introduction To Safety Issues In Biosimilars/Follow-On Biopharmaceuticals”, J. Med. CBR Def., 7:1-18, (2009); Roger, “Biosimilars: current status and future directions,” Expert Opin. Biol. Ther., 10(7):1011-1018 (2010)). One exemplary report states that “[t]he size and complexity of . . . therapeutic proteins make the production of an exact replica almost impossible; therefore, there are no true generic forms of these proteins . . . . Verification of the similarity of biosimilars to innovator medicines remains a key challenge” (Hincal, supra). This disclosure provides, in part, methods and compositions sufficient to make and test products that qualify as bevacizumab, e.g., that are interchangeable versions of Avastin®.

Glycoprotein preparations can be obtained from any source. In some instances, providing or obtaining a glycoprotein preparation (e.g., such as a glycoprotein drug substance or a precursor thereof), e.g., that is or includes a glycoprotein, can include providing a host cell, e.g., a mammalian host cell (e.g., a CHO cell) that is genetically engineered to express a glycoprotein having an amino acid sequence at least 85% identical to SEQ ID NO:1 and an amino acid sequence at least 85% identical to SEQ ID NO:2 (e.g., a genetically engineered cell); culturing the host cell under conditions suitable to express the glycoprotein (e.g., mRNA and/or protein); and, optionally, purifying the expressed glycoproteins, e.g., in the form of a recombinant antibody) from the cultured cell, thereby producing a glycoprotein preparation. In some instances, the host cell is genetically engineered to express a glycoprotein having an amino acid sequence at least 90%, 91%, 92%, 93%, 94%, 95%, 96%, 97%, 98%, 99%, or 100% identical to SEQ ID NO:1 and an amino acid sequence at least 90%, 91%, 92%, 93%, 94%, 95%, 96%, 97%, 98%, 99%, or 100% identical to SEQ ID NO:2, wherein the expressed amino acid sequences form a recombinant antibody composition.

As used herein percent (%) sequence identity with respect to a sequence is defined as the percentage of amino acid residues or nucleotides in a candidate sequence that are identical with the amino acid residues or nucleotides in the reference sequence, after aligning the sequences and introducing gaps, if necessary, to achieve the maximum percent sequence identity. (E.g., gaps can be introduced in one or both of a first and a second amino acid or nucleic acid sequence for optimal alignment and non-homologous sequences can be disregarded for comparison purposes). Alignment for purposes of determining percent sequence identity can be achieved in various ways that are within the skill in the art, for instance, using publicly available computer software such as BLAST, ALIGN or Megalign (DNASTAR) software. Those skilled in the art can determine appropriate parameters for measuring alignment, including any algorithms needed to achieve maximal alignment over the full length of the sequences being compared. In one embodiment, the length of a reference sequence aligned for comparison purposes is at least 30%, e.g., at least 40%, e.g., at least 50%, 60%, 70%, 80%, 90%, or 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. In some instances a product will include amino acid variants, e.g., species that differ at terminal residues, e.g., at one or two terminal residues. In instances of such cases the sequence identity which is compared is the identity between the primary amino acid sequences of the most abundant active species in each of the products being compared. In some instances sequence identity refers to the amino acid sequence encoded by a nucleic acid that can be used to make the product.

In some instances, a bevacizumab signature disclosed herein can include 1, 2, 3, 4, 5, 6, 7, 8, 9, 10, 11, 12, 13, 14, or 15 of the bevacizumab parameters (e.g., the reference criterion therefor) shown in Table 1 (e.g., including any combination of 2 or more (e.g., 3, 4, 5, 6, 7, 8, 9, 10, 11, 12, 13, 14, or 15) of parameter numbers 1-15 shown in Table 1).

In some instances, a bevacizumab signature disclosed herein can include, other structures or characteristics (whether intrinsic or extrinsic) of bevacizumab, e.g., that distinguish bevacizumab from non-bevacizumab glycoprotein (see application entitled Methods of Evaluating and Making Biologics, filed on Jun. 1, 2012, as U.S. Ser. No. 61/654,467, for exemplary structures or characteristics). Examples of structures or characteristics include: the amount of GalNAc in the preparation (e.g., relative to total glycans of the preparation); the amount of truncated core glycans; the amount of aglycosylated glycans; the amount of each species of high mannose glycans; the amount of sialylated glycans or particular species of sialylated glycans; the ratio of monosialylated:diasylated glycans, the amount of diacetylated sialic acids (NeuXAc2), the amount of one or more of: NeuGc; NeuAc; Neu5,7,Ac2; Neu5Gc,9Ac; Neu5,8Ac2; Neu5,9Ac2; Neu4,5Ac2. Examples of parameters related to the glycan linkage composition of a glycoprotein preparation can be: the presence or amount of one or more of terminal fucose; terminal mannose; terminal galactose; 2 linked mannose; 3.6 linked mannose; terminal GlcNAc; terminal GalNAc; 4 linked GlcNAc; 4.6 linked GlcNAc. A parameter may also be the ratio of one of these to another or to another property. Examples of parameters related to the glycoform composition of a glycoprotein preparation include: the absence or presence of one or more specific glycoforms (e.g., one or more glycoforms described in Table 1); the amount or abundance of a specific glycoform in the preparation relative to total glycoforms (e.g., in a w/w basis); the ratio of one particular glycoform to another. Examples of parameters related to post-translational modification in the preparation include: the absence or presence of one or more specific post-translational modification; the abundance or distribution of one or more specific post-translational modification. In some instances, the present disclosure includes determining whether information evaluated for a glycoprotein preparation meets a bevacizumab signature, e.g., by comparing the information with the bevacizumab signature and/or confirming that the information has a defined (e.g., predefined) relationship with the bevacizumab signature.

In some instances, methods disclosed herein can be used to confirm the identity and/or quality of bevacizumab preparations. For example, methods can include assessing preparations (e.g., samples, lots, and/or batches) of a test glycoprotein to confirm whether the test glycoprotein qualifies as bevacizumab, and, optionally, qualifying the test protein as bevacizumab if qualifying criteria (e.g. predefined qualifying criteria) are met; thereby evaluating, identifying, and/or producing (e.g., manufacturing) bevacizumab.

Methods of the disclosure have a variety of applications and include, e.g., quality control at different stages of manufacture, analysis of bevacizumab preparations prior to or after completion of manufacture (e.g., prior to or after distribution to a fill/finish environment or facility), prior to or after release into commerce (e.g., before distribution to a pharmacy, a caregiver, a patient, or other end-user). Thus, the preparation can be any preparation that potentially comprises bevacizumab. In an embodiment the bevacizumab preparation is a drug substance (an active pharmaceutical ingredient or “API”) or a drug product (an API formulated for use in a subject such as a human patient). In an embodiment the preparation is from a stage of manufacture or use that is prior to release to care givers or other end-users; prior to packaging into individual dosage forms, such as syringes, pens, vials, or multi-dose vials; prior to determination that the batch can be commercially released, prior to production of a Certificate of Testing, Material Safety Data Sheet (MSDS) or Certificate of Analysis (CofA) of the preparation. In an embodiment the glycoprotein preparation from an intermediate step in production, e.g., it is after secretion of the glycoprotein from a cell but prior to purification of drug substance.

Evaluations from methods of the invention are useful for guiding, controlling or implementing a number of activities or steps in the process of making, distributing, and monitoring and providing for the safe and efficacious use of bevacizumab. Thus, in an embodiment, e.g., responsive to the evaluation, e.g., depending on whether a criterion is met, a decision or step is taken. The method can further comprise one or both of the decision to take the step and/or carrying out the step itself. E.g., the step can comprise one in which the preparation (or another preparation for which the preparation is representative) is: classified; selected; accepted or discarded; released or processed into a drug product; rendered unusable for commercial release, e.g., by labeling it, sequestering it, or destroying it; passed on to a subsequent step in manufacture; reprocessed (e.g., the preparation may undergo a repetition of a previous process step or subjected to a corrective process); formulated, e.g., into drug substance or drug product; combined with another component, e.g., an excipient, buffer or diluent; disposed into a container; divided into smaller aliquots, e.g., unit doses, or multi-dose containers; combined with another preparation of bevacizumab; packaged; shipped; moved to a different location; combined with another element to form a kit; combined, e.g., placed into a package with a delivery device, diluent, or package insert; released into commerce; sold or offered for sale; delivered to a care giver or other end-user; or administered to a subject. E.g., based on the result of the determination or whether one or more subject entities is present, or upon comparison to a reference standard, the batch from which the preparation is taken can be processed, e.g., as just described.

Methods described herein may include making a decision: (a) as to whether a preparation may be formulated into drug substance or drug product; (b) as to whether a preparation may be reprocessed (e.g., the preparation may undergo a repetition of a previous process step); or (c) that the preparation is not suitable for formulation into drug substance or drug product. In instances the method comprises: formulating as referred to in step (a), reprocessing as referred to in step (b), or rendering the preparation unusable for commercial release, e.g., by labeling it or destroying it, as referred to in step (c).

### Parameter Evaluation

The amino acid sequence of the heavy chain of bevacizumab (Avastin®) is disclosed herein as SEQ ID NO:1. The amino acid sequence of the light chain of bevacizumab (Avastin®) is disclosed herein as SEQ ID NO:2.

Parameters disclosed herein can be analyzed by any available suitable method. In some instances, glycan structure and composition as described herein are analyzed, for example, by one or more, enzymatic, chromatographic, mass spectrometry (MS), chromatographic followed by MS, electrophoretic methods, electrophoretic methods followed by MS, nuclear magnetic resonance (NMR) methods, and combinations thereof. Exemplary enzymatic methods include contacting a glycoprotein preparation with one or more enzymes under conditions and for a time sufficient to release one or more glycans (e.g., one or more exposed glycans). In some instances, the one or more enzymes includes PNGase F. Exemplary chromatographic methods include, but are not limited to, Strong Anion Exchange chromatography using Pulsed Amperometric Detection (SAX-PAD), liquid chromatography (LC), high performance liquid chromatography (HPLC), ultra performance liquid chromatography (UPLC), thin layer chromatography (TLC), amide column chromatography, and combinations thereof. Exemplary mass spectrometry (MS) include, but are not limited to, tandem MS, LC-MS, LC-MS/MS, matrix assisted laser desorption ionisation mass spectrometry (MALDI-MS), Fourier transform mass spectrometry (FTMS), ion mobility separation with mass spectrometry (IMS-MS), electron transfer dissociation (ETD-MS), and combinations thereof. Exemplary electrophoretic methods include, but are not limited to, capillary electrophoresis (CE), CE-MS, gel electrophoresis, agarose gel electrophoresis, acrylamide gel electrophoresis, SDS-polyacrylamide gel electrophoresis (SDS-PAGE) followed by Western blotting using antibodies that recognize specific glycan structures, and combinations thereof. Exemplary nuclear magnetic resonance (NMR) include, but are not limited to, one-dimensional NMR (1D-NMR), two-dimensional NMR (2D-NMR), correlation spectroscopy magnetic-angle spinning NMR (COSY-NMR), total correlated spectroscopy NMR (TOCSY-NMR), heteronuclear single-quantum coherence NMR (HSQC-NMR), heteronuclear multiple quantum coherence (HMQC-NMR), rotational nuclear overhauser effect spectroscopy NMR (ROESY-NMR), nuclear overhauser effect spectroscopy (NOESY-NMR), and combinations thereof.

In some instances, techniques described herein may be combined with one or more other technologies for the detection, analysis, and or isolation of glycans or glycoproteins. For example, in certain instances, glycans are analyzed in accordance with the present disclosure using one or more available methods (to give but a few examples, see Anumula, Anal. Biochem. 350(1):1, 2006; Klein et al., Anal. Biochem., 179:162, 1989; and/or Townsend, R.R. Carbohydrate Analysis” High Performance Liquid Chromatography and Capillary Electrophoresis., Ed. Z. El Rassi, pp 181-209, 1995, each of which is incorporated herein by reference in its entirety). For example, in some instances, glycans are characterized using one or more of chromatographic methods, electrophoretic methods, nuclear magnetic resonance methods, and combinations thereof.

In some instances, methods for evaluating one or more bevacizumab-specific parameters, e.g., in a glycoprotein preparation, e.g., one or more of bevacizumab parameters disclosed in Table 1 in a glycoprotein preparation are known in the art and/or are disclosed in Table 2:

TABLE 2

| Method(s) | Relevant literature | Parameter |
|---|---|---|
| C18 UPLC Mass | Chen and Flynn, Anal. | Glycan(s) |
| Spec.* | Biochem., 370: 147-161 (2007) | (e.g., N-linked glycan, exposed N- |
| | Chen and Flynn, J. Am. Soc. | linked glycan, glycan detection, glycan |
| | Mass Spectrom., 20: 1821-1833 | identification, and characterization; |
| | (2009) | site specific glycation; glycoform |
| | | detection (e.g., parameters 1-7); |
| | | percent glycosylation; and/or |
| | | aglycoosyl) |
| Peptide LC-MS | Dick et al., Biotechnol. Bioeng., | C-terminal lysine (e.g., parameter 8) |
| (reducing/non- | 100: 1132-1143 (2008) | |
| reducing) | | |
| LC-MS | Dick et al., Biotechnol. Bioeng., | |
| (reducing/non- | 100: 1132-1143 (2008) | |
| reducing/alkylated) | | |
| Weak cation | Dick et al., Biotechnol. Bioeng., | |
| exchange (WCX) | 100: 1132-1143 (2008) | |
| chromatography | | |
| Peptide LC-MS | Chelius et al., Anal. Chem., | |
| (reducing/non- | 78: 2370-2376 (2006) | |
| reducing) | | |
| Peptide LC-MS | Yan et al., J. Chrom. A., | Methionine oxidation (e.g., parameter |
| (reducing/non- | 1164: 153-161 (2007); | 11) |
| reducing) | Xie et al., mAbs, 2: 379-394 | |
| | (2010) | |
| Peptide LC-MS | Miller et al., J. Pharm. Sci., | Site specific glycation (e.g., parameter |
| (reducing/non- | 100: 2543-2550 (2011) | 12) |
| reducing) | | |
| Peptide LC-MS | Wang et al., Anal. Chem., | Free cysteine (e.g., parameters 13-15) |
| (reducing/non- | 83: 3133-3140 (2011); | |
| reducing) | Chumsae et al., Anal. Chem., | |
| | 81: 6449-6457 (2009) | |

Literature shown in Table 2 are hereby incorporated by reference in their entirety or, in the alternative, to the extent that they pertain to one or more of the methods disclosed in Table 2.

### EXAMPLES

### Example 1

### Characterization of Bevacizumab

Avastin® samples were analyzed to determine the amino acid sequences of the heavy and light chains of the bevacizumab antibody. The sequence of the heavy chain is shown as SEQ ID NO:1 and the sequence of the light chain is shown as SEQ ID NO:2.

Characterization of Avastin® was performed by orthogonal methods. Measurements made included use of glycan profiling, glycoform analysis, post-translational modification analysis, and analysis of other intrinsic and extrinsic structures or features. Of 113 Avastin®/bevacizumab structures or features that were measured or determined, 15 were determined to be bevacizumab parameters, i.e., parameters of bevacizumab that distinguish bevacizumab from non-bevacizumab antibody products. These 15 bevacizumab parameters and values are listed in Table 3 for an illustrative sample of bevacizumab.

TABLE 3

| Parameter | Parameter | |
|---|---|---|
| No. | Category¹ | Value² |
| 1 | HM5 | 0.71 |
| 2 | HM6 | 0.01 |
| 3 | HM7 | 0.01 |
| 4 | G0F | 68.57 |
| 5 | G1F | 16.68 |
| 6 | G1F | 5.26 |
| 7 | G2F | 2.24 |
| 8 | C-terminal-K | 2.00 |
| 9 | HC-Pyroglu | 1.90 |
| 10 | LC-pyroglu | 0.00 |
| 11 | HC-M256- | 6.10 |
| | Sulfo | |
| 12 | LC-K149- | 1.00 |
| | Glyc | |
| 13 | LC135 | 1.00 |
| 14 | HC148 | 6.10 |
| 15 | HC204 | 3.20 |

¹Detailed descriptions of the structures/features of each parameter are provided in Table 1.
²See Table 1 for unit information.

The information (values) shown for each bevacizumab parameter in Table 3 were used to formulate a reference criterion or rule for each bevacizumab parameter (shown in Table 1).

### Example 2

### Qualification Glycoprotein Preparations

The reference criterion or rules described in Table 1 were used to determine whether blinded samples qualify as bevacizumab. Multiple glycoprotein products were prepared and samples thereof were blinded for identity analysis (samples A and B).

Sample A was analyzed and values were obtained for each of the bevacizumab parameters in Table 1. The values of these parameters in sample A are presented in Table 4 below. In addition, values obtained for sample A were compared to the reference criteria for bevacizumab as shown in Table 4:

TABLE 4

| | | | | Comparison |
|---|---|---|---|---|
| | | | | of “A” |
| | | | | Values and |
| Parameter | Parameter | Sample “A” | Reference | reference |
| No. | category¹ | Value | Criterion² | criterion |
| 1 | HM5 | 3.1 | <0.90% | |
| 2 | HM6 | 2.59 | <0.05% | |
| 3 | HM7 | 1.18 | <0.05% | |
| 4 | G0F | 45.64 | >60.00% | |
| 5 | G1F | 22.83 | <20.00% | |
| 6 | G1F | 5.90 | <6.00% | |
| 7 | G2F | 3.47 | <2.60% | |
| 8 | C-terminal-K | 45.20 | <5.00% | |
| 9 | HC-Pyroglu | 100.0 | <10.00% | |
| 10 | LC-pyroglu | 70.00 | <3.00% | |
| 11 | HC-M256- | 5.50 | >4.00% | ✓ |
| | Sulfo | | | |
| 12 | LC-K149- | 0.90 | >0.50% | ✓ |
| | Glyc | | | |
| 13 | LC135 | 2.00 | <1.20% | |
| 14 | HC148 | 13.40 | <10.00% | |
| 15 | HC204 | 4.10 | <5.00% | |

¹Detailed descriptions of the structures/features of each parameter are provided in Table 1.
²See Table 1 for unit information.
✓ Illustrates that a value meets the reference criterion/rule.

Data plotted in Table 4 confirms that sample A is not bevacizumab according to the methods described herein. Based on these data, sample A does not meet a bevacizumab signature that comprises all 15 parameters and, thus, does not qualify as bevacizumab.

Sample B was analyzed and values were obtained for each of the bevacizumab parameters in Table 1. The values of these parameters in sample B are presented in Table 5 below. In addition, values obtained for sample B were compared to the reference criteria for bevacizumab as shown in Table 5:

TABLE 5

| | | | | Comparison |
|---|---|---|---|---|
| | | | | of sample |
| | | | | “B” Values |
| | | | | and |
| Parameter | Parameter | Sample “B” | Reference | reference |
| No. | Category¹ | Value | Criterion² | criterion |
| 1 | HM5 | 0.72 | <0.90% | ✓ |
| 2 | HM6 | 0.01 | <0.05% | ✓ |
| 3 | HM7 | 0.01 | <0.05% | ✓ |
| 4 | G0F | 68.46 | >60.00% | ✓ |
| 5 | G1F | 16.84 | <20.00% | ✓ |
| 6 | G1F | 4.80 | <6.00% | ✓ |
| 7 | G2F | 2.26 | <2.60% | ✓ |
| 8 | C-terminal-K | 1.60 | <5.00% | ✓ |
| 9 | HC-Pyroglu | 2.30 | <10.00% | ✓ |
| 10 | LC-pyroglu | 0.00 | <3.00% | ✓ |
| 11 | HC-M256- | 5.90 | >4.00% | ✓ |
| | Sulfo | | | |
| 12 | LC-K149- | 0.40 | >0.50% | ✓ |
| | Glyc | | | |
| 13 | LC135 | 0.70 | <1.20% | ✓ |
| 14 | HC148 | 5.60 | <10.00% | ✓ |
| 15 | HC204 | 1.90 | <5.00% | ✓ |

¹Detailed descriptions of the structures/features of each parameter are provided in Table 1.
²See Table 1 for unit information.
✓ Illustrates that a value meets the reference criterion/rule.

As shown in Table 5, sample B meets all listed reference criteria signatures for bevacizumab. Accordingly, sample B does meet a bevacizumab signature that includes all 15 parameters and, thus, qualifies as bevacizumab.

While the methods have been described in conjunction with various instances and examples, it is not intended that the methods be limited to such instances or examples. On the contrary, the methods encompass various alternatives, modifications, and equivalents, as will be appreciated by those of skill in the art.

1-28. (canceled)

29. A method of manufacturing a bevacizumab drug product, comprising:
providing or obtaining a test glycoprotein preparation;
acquiring at least one value for a bevacizumab parameter listed in Table 1 for the test glycoprotein preparation; and
processing at least a portion of the test glycoprotein preparation as bevacizumab drug product if the at least one value for the test glycoprotein preparation meets a reference criterion shown in Table 1 for the parameter,
thereby manufacturing a bevacizumab drug product.

30. The method of claim 29, comprising:
acquiring values for any combination of two or more bevacizumab parameters listed in Table 1; and
processing at least a portion of the test glycoprotein preparation as bevacizumab drug product if the values for the any combination of two or more bevacizumab parameters for the test glycoprotein preparation meet the corresponding reference criterion shown in Table 1 for the parameters.

31. The method of claim 30, wherein the any combination of two or more bevacizumab parameters comprises:
2, 3, 4, 5, 6, 7, 8, 9, 10, 11, 12, 13, 14, 15, all, or a plurality of the bevacizumab parameters listed in Table 1; or
any two or more of parameter numbers 1, 2, 3, 4, 5, 6, 7, 8, 9, 10, 11, 12, 13, 14, and/or 15 shown in Table 1.

32. The method of claim 29, wherein the test glycoprotein preparation comprises a recombinant antibody composition having a first amino acid sequence with at least 85% identity to SEQ ID NO:1 (e.g., 90, 95, 98, or 100% identity to SEQ ID NO:1) and a second amino acid sequence with at least 85% identity to SEQ ID NO:2 (e.g., 90, 95, 98, or 100% identity to SEQ ID NO:2).

33. The method of claim 32, wherein the first and second amino acid sequences form a recombinant antibody.

34. The method of claim 29, wherein the test glycoprotein preparation comprises a recombinant antibody composition having a first amino acid sequence with 100% identity to SEQ ID NO:1 and a second amino acid sequence with 100% identity to SEQ ID NO:2.

35. The method of claim 34, wherein the first and second amino acid sequences form a recombinant antibody.

36. The method of claim 29, further comprising:
after the step of acquiring the value(s) and before the step of processing, obtaining a plurality of assessments made by comparing the value(s) with a corresponding reference criterion shown in Table 1.

37. The method of claim 29, wherein at least one value is directly obtained by performing an analytical test on the test antibody or glycoprotein preparation.

38. The method of claim 37, wherein the value is directly obtained using a method provided in Table 2.

39. The method of claim 29, wherein the processing step comprises combining the test antibody preparation with an excipient or buffer.

40. The method of claim 29, wherein the processing step comprises one or more of: formulating the test protein preparation; processing the test protein preparation into a drug product; combining the test protein preparation with a second component, e.g., an excipient or buffer; changing the concentration of the test protein in the preparation; lyophilizing the test protein preparation; combining a first and second aliquot of the test protein to provide a third, larger, aliquot; dividing the test protein preparation into smaller aliquots; disposing the test protein preparation into a container, e.g., a gas or liquid tight container; packaging the test protein preparation; associating a container comprising the test protein preparation with a label (e.g., labeling); shipping or moving the test protein preparation to a different location.

41. The method of claim 29, wherein the processed drug product or antibody is approved under Section 351(k) of the Public Health Service (PHS) Act.

42. The method of claim 29, wherein the processed drug product or antibody is not approved under biologics license application (BLA) under Section 351(a) of the Public Health Service (PHS) Act.

43. The method of claim 29, wherein the value for the test glycoprotein preparation comprises an average (e.g., mean) of a range of values for the parameter for multiple (e.g., 2, 3, 4, 5, 10, 15, 20, or more) batches or samples of the target protein.

44. The method of claim 29, wherein one or more, including all, of the reference criterion shown in Table 1 is/are a specification for commercial release of a drug product under Section 351(k) of the Public Health Service (PHS) Act.

45. The method of claim 29, wherein the value is acquired for one, two, or more samples or batches.

46. A method of manufacturing a bevacizumab drug product, comprising:
providing a host cell that is genetically engineered to express a first amino acid sequence having a sequence with at least about 85% identity to SEQ ID NO:1 (e.g., 90, 95, 98, or 100% identity to SEQ ID NO:1) and a second amino acid sequence having a sequence with at least about 85% identity to SEQ ID NO:2 (e.g., 90, 95, 98, or 100% identity to SEQ ID NO:2), wherein the expressed amino acid sequences form a recombinant antibody composition,
culturing the host cell under conditions whereby the cell expresses the first and second amino acid sequences, wherein the expressed first and second amino acid sequences form recombinant antibodies,
harvesting the recombinant antibodies from the host cell culture to produce an antibody preparation,
acquiring a value for each parameter listed in Table 1 for the antibody preparation; and
processing at least a portion of the antibody preparation into bevacizumab drug product if the value for each parameter listed in Table 1 for the antibody preparation meets the reference criterion shown in Table 1,
thereby manufacturing a bevacizumab drug product.

47. The method of claim 46, comprising:
providing a host cell that is genetically engineered to express a first amino acid sequence having the sequence of SEQ ID NO:1 and a second amino acid sequence having the sequence of SEQ ID NO:2, wherein the expressed amino acid sequences form a recombinant antibody composition,
culturing the host cell under conditions whereby the cell expresses the first and second amino acid sequences, wherein the expressed first and second amino acid sequences form recombinant antibodies,
harvesting the recombinant antibodies from the host cell culture to produce an antibody preparation,
acquiring at least one value for a bevacizumab parameter listed in Table 1 for the antibody preparation; and
processing or directing the processing of at least a portion of the antibody preparation as bevacizumab drug product if the at least one value for the antibody preparation meets a reference criterion shown in Table 1,
thereby manufacturing a bevacizumab drug product.

48. The method of claim 46, comprising:
acquiring values for any combination of 2, 3, 4, 5, 6, 7, 8, 9, 10, 11, 12, 13, 14, 15, or all, or a plurality of the bevacizumab parameters listed in Table 1; and
processing at least a portion of the test glycoprotein preparation as bevacizumab drug product if the values for the any combination of two or more bevacizumab parameters for the test glycoprotein preparation meet the corresponding reference criterion shown in Table 1 for the parameters.


SEQUENCE LISTING (2 sequences)

SEQ ID NO: 1 - protein, 453 aa, Artificial Sequence
  Description of Artificial Sequence Synthetic polypeptide
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 214 aa, Artificial Sequence
  Description of Artificial Sequence Synthetic polypeptide
DIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC